(19) United States
(12) Patent Application Publication
(10) Pub. No.: US 2006/0025798 A1
(43) Pub. Date: Feb. 2, 2006

(54) Method and apparatus for fecal continence

(76) Inventors: Timothy C. Cook, Wayzata, MN (US); Richard A. Noddin, Elk River, MN (US); John H. Burton, Minnetonka, MN (US)

Correspondence Address: Schwegman, Lundberg, Woessner & Kluth, P.A., P.O. Box 2938, Minneapolis, MN, 55402, US

(21) Appl. No.: 11/063,229

(22) Filed: Feb. 22, 2005

Related U.S. Application Data
(60) Provisional application No. 60/546,769, filed on Feb. 23, 2004.

(51) Int. Cl. A61M 29/00

(52) U.S. Cl. 606/192

(57) ABSTRACT

Apparatus and method for adjustably restricting a selected body lumen such as an anal canal of a patient to passively treat fecal incontinence. The apparatus in various examples comprises a plurality of expandable elements. Each of the elements is comprised of a membrane of biocompatible material enclosing a volume. The elements expand upon introduction of fluid into the elements and contract upon withdrawal of fluid from the elements. The expandable elements are adjustable in size by adjusting an amount of fluid in the elements. Fluid communication to the expandable elements is provided by a plurality of flexible conduits of biocompatible material coupled to both the expandable elements and a fill port block comprising a plurality of fill ports.

### RELATED APPLICATIONS

This application claims the benefit under 35 U.S.C. 119(e) of Provisional Patent Application Ser. No. 60/546,769 filed Feb. 23, 2004, the entire specification of which is hereby incorporated by reference.

### TECHNICAL FIELD

This document relates generally to implantable devices, and in particular to method and apparatus for adjustably restricting a selected body lumen, such as an anal canal of a patient to passively treat fecal incontinence.

### BACKGROUND

Various implantable devices are known in which distensible medical devices are implanted into the tissue of a human to treat fecal incontinence. These devices have typically relied upon a pump activated by a patient to restrict or constrict the anal canal of the patient to maintain continence. However, patients have been known to have difficulty activating such devices and properly controlling them.

There is a need in the art for a continence device that does not require patient activation and which assists in restoring natural anatomical function. Such a device should be adjustable by a physician after implantation.

### SUMMARY

This document discusses implantable medical apparatus and method for providing fecal continence. The embodiments of the present subject matter include a plurality of expandable elements. Each of the elements includes a membrane of biocompatible material enclosing a volume. The elements expand upon introduction of fluid into the elements and contract upon withdrawal of fluid from the elements. The expandable elements are adjustable in size by adjusting an amount of fluid in the elements. Fluid communication to the expandable elements is provided by a plurality of flexible conduits of biocompatible material coupled to both the expandable elements and a fill port block comprising a plurality of fill ports.

Some embodiments of the present subject matter are adapted to provide control of a body lumen by, among other things, locating a plurality of expandable elements in a cluster around the body lumen of a patient, establishing fluid communication with the expandable elements, and adjusting volume of the elements to restrict the body lumen. In applications where the body lumen is the anal canal, it is possible to restore fecal continence to patients where the expandable elements are properly adjusted.

This summary is intended to provide an overview of the subject matter of the present application and is not intended to be an exclusive or exhaustive explanation of the present subject matter. The reader is directed to the detailed description to provide further information about the subject matter of the present patent application.

### BRIEF DESCRIPTIONS OF THE DRAWINGS

In the drawings like numerals refer to like components throughout the several views.

FIG. 1 is a drawing of an apparatus according to one embodiment of the present subject matter.

FIG. 2 is a drawing of an apparatus according to one embodiment of the present subject matter.

FIGS. 3A, 3B, and 3C are illustrations of one embodiment of the apparatus as implanted in one example of an application of the present subject matter.

FIG. 4 is a drawing of an apparatus according to one embodiment of the present subject matter.

FIG. 5 is a drawing of an apparatus according to one embodiment of the present subject matter.

FIG. 6 is a drawing of an apparatus according to one embodiment of the present subject matter.

FIG. 7 is a drawing of an apparatus according to one embodiment of the present subject matter.

FIG. 8 is a flowchart of a method for providing fecal continence according to one embodiment of the present subject matter.

### DETAILED DESCRIPTION

In the following detailed description, reference is made to the accompanying drawings which form a part hereof, and in which is shown by way of illustration specific embodiments in which the invention may be practiced. It is to be understood that other embodiments may be utilized and structural changes may be made without departing from the scope of the present invention.

This document discusses an implantable apparatus to provide a controllable restriction about a body lumen. One application of the present subject matter is for restoring control fecal continence. The present apparatus offers a passive system in that intervention by the user is not necessary for its use. However, the system is adapted to be postoperatively adjustable. In so doing, the system can be adjusted to restore natural function to a body lumen. In applications involving fecal incontinence, placement of the system about the anal canal and proper adjustment may provide substantially normal anatomical function to a patient.

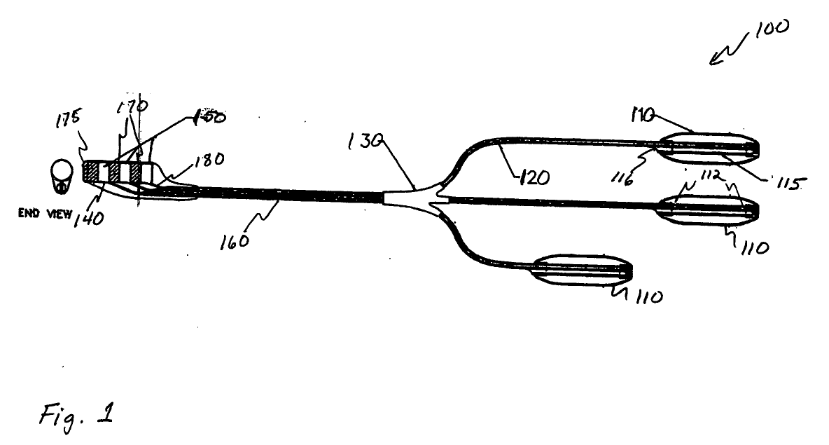

FIG. 1 is a drawing of an apparatus according to one embodiment of the present subject matter. Such a device may be implanted and adjusted to controllably restrict flow through a body lumen. In applications involving restoration of fecal continence, the apparatus 100 includes a plurality of expandable elements or balloons 110. The balloons 110 include an outer wall constructed of a biocompatible resiliently elastomeric polymer or polymer blend of polyurethane, silicone, or the like. Some examples of the construction of implantable expandable elements are provided in U.S. Pat. No. 5,964,806, to Cook et al., which is incorporated herein by reference in its entirety. The balloons 110 expand when filled with fluid and contract when fluid is withdrawn. In one embodiment, each balloon encloses a volume of about 12 cubic centimeters. The embodiment shown includes three balloons 110. A lumen segment 115 within the elements 110 contains holes 112 to introduce the fluid into the elements 110. Conduit 120 couples the distal ends of the balloons 110 to a fill port block 140. In one embodiment conduit 120 is comprised of silicone tubing. In this embodiment the cavity of the fill port block 140 has a tube shape and the ports 150 are disposed serially along the tube. Sealing plugs 170 are positioned along the tube to isolate the ports 150. The third sealing plug 175 has a mushroom shape to cap the fill port block 140. The sealing plugs 170, 175 are self-sealing to allow a fill needle to be inserted through a plug 170, 175 to adjust the level of fluid in each balloon 110. The plugs 170, 175 re-seal when the needle is withdrawn. By isolating each port 150, each balloon 110 is independently adjustable. Conduit sleeve 130 provides strain relief for the conduit/port junctions 180. The conduit sleeve is comprised of a biocompatible material such as molded silicone. Conduit segment 160 is a three lumen segment. In one embodiment, segment 160 is a bundle of three of the single conduit segments 120 passed through the conduit sleeve 130. In another embodiment, the single conduit segments 120 and the three conduit segment 160 terminate at the conduit sleeve 130. Other embodiments with various different arrangements are possible without departing from the scope of the present subject matter.

In one embodiment of the apparatus 100, the balloons include a lumen segment 115 comprising two lumen tubing. A first lumen is attached to the conduit 120 and contains holes 112 to introduce fluid into the balloons 110. A second lumen 116 is shaped to receive a push-wire to facilitate placement and/or tunneling of the balloons 110 into position.

The fill port block 140 is implanted in a manner to allow access to the fill ports 150. In some applications, fill port block 140 is implanted subcutaneously to provide ready adjustment of the device using a syringe and needle. In applications involving restriction of the anal canal, filling the balloons 110 with fluid provides pressure to support and restrict the anal canal wall.

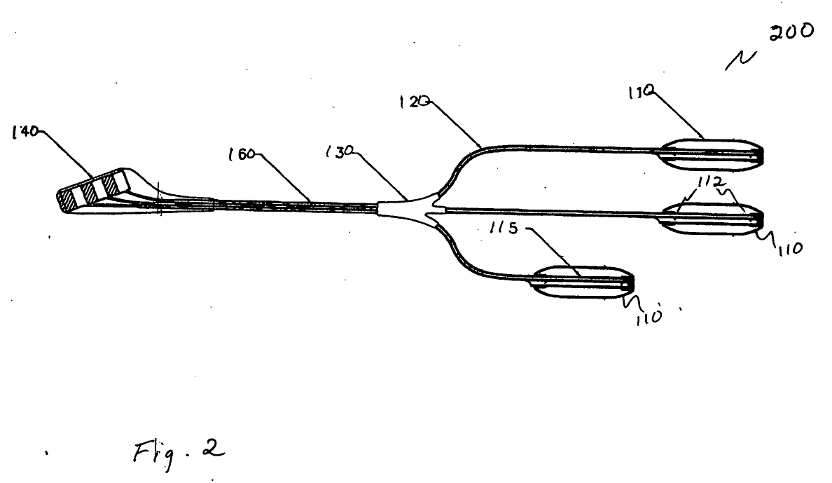

FIG. 2 shows one embodiment of the present subject matter including an apparatus 200 with the fill port block 140 oriented at a different angle to the tubing than the fill port block 140 in FIG. 1. The example in FIG. 2 may be advantageous in implantation to match contours of a patient's body. Other angles and configurations are possible without departing from the scope of the present subject matter.

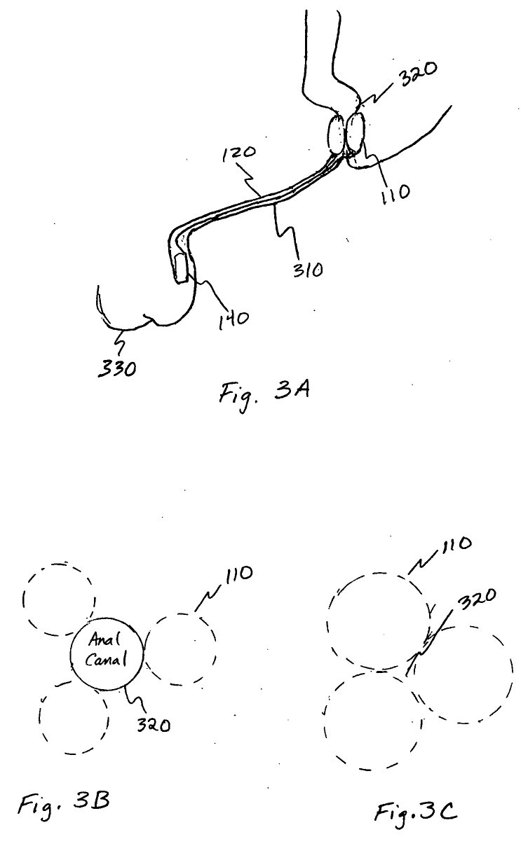

FIGS. 3A, 3B and 3C are illustrations of one embodiment of the apparatus as implanted in one example of an application of the present subject matter. To implant the device in a male patient, the patient is placed in a modified lithotomy position with the perineal area 310 exposed. An incision is made in a midline of the perineal area 310 equidistant between the posterior side of the scrotum 330 and anterior side of the rectum. A delivery tool is then used to tunnel interiorly towards the scrotal area to place the fill port block 140 just under the skin in the patient's scrotum 330. A delivery tool is used to capture a balloon 110 and tunnel in a posterior manner towards the rectal and sphincter area. If the embodiment of the apparatus used contains a second lumen 116, the delivery tool could be a push-wire inserted into the lumen 116. Three balloons 110 are placed outside of the external sphincter tissue in a cluster somewhat resembling a tulip shape as is shown in FIGS. 3B and 3C. Because the external sphincter tissue is striated and wraps the anal canal 320, expanding the balloon cluster will provide support to allow the anal canal 320 to close in; thereby passively providing fecal continence. Access to the fill port block 140 allows the volume of the balloons to be adjusted post-operatively.

To implant the device in a female patient, the patient is also placed in a modified lithotomy position with the perineal area exposed. An incision is made in the midline of the perineal area and equidistant from the most posterior portion of the introitus and the anterior side of the rectum. Using a dilator tool a balloon 110 is pushed through the dilator to the external sphincter tissue. Three balloons 110 are again placed in a cluster outside the external sphincter tissue. The fill port is placed at the perineal incision, just under the skin.

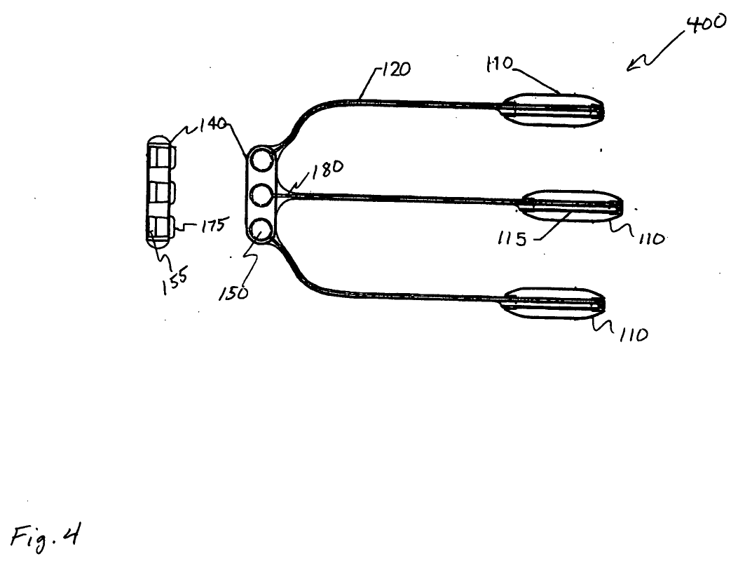

FIG. 4 shows an apparatus according to one embodiment of the present subject matter. In the demonstrated embodiment of apparatus 400, fill ports 150 are disposed in parallel along the fill port block 140. The single lumen conduit 120 is attached substantially perpendicular to the fill port block 140 and the block 140 provides strain relief to the tube/port junctions 180. Each fill port 150 has a mushroom shaped self-sealing plug 175 to isolate the port. In one embodiment of fill port block 140, the block 140 comprises machined titanium or tantalum. In another embodiment, the block 140 comprises drawn titanium or tantalum. In a further embodiment of block 140, the fill ports 150 contain a fill port can 155. The fill port can 155 lines the fill port and is comprised of a metal such as titanium or tantalum, for example, and provides protection against a fill needle going beyond a fill port 150.

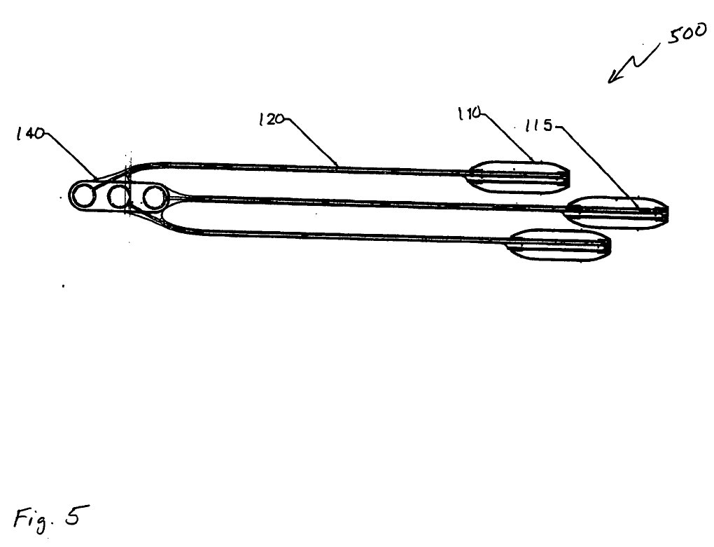

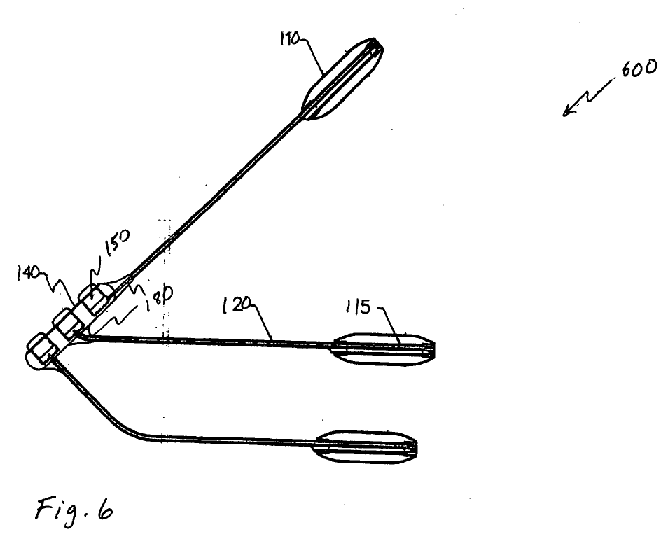

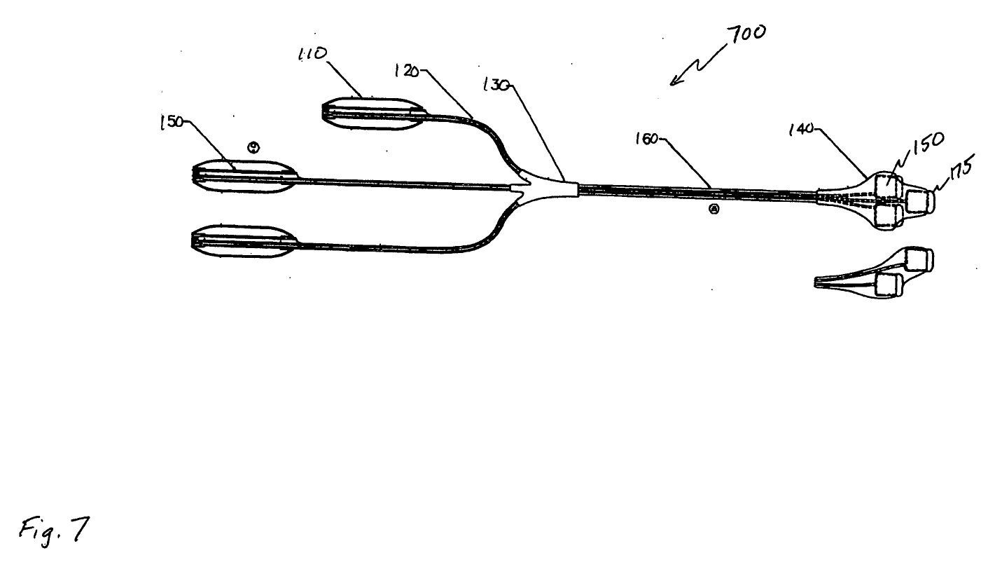

FIG. 5 is a drawing of an apparatus according to one embodiment of the present subject matter. In this embodiment, the orientation of the fill port block 140 of apparatus 500 is achieved by rotating the fill port block 140 in FIG. 4 ninety degrees. The conduit 120 is attached substantially parallel to a length of the fill port block. FIG. 6 is a drawing of an apparatus according to one embodiment of the present subject matter. The apparatus 600 shown is a combination of the parallel and perpendicular orientations. Some conduit/port junctions 180 are parallel to the fill port block 140 and some junctions 180 are perpendicular. FIG. 7 is a drawing of an apparatus according to one embodiment of the present subject matter. The demonstrated embodiment includes an apparatus 700 including fill ports 150 staggered instead of aligned. Thus, the present subject matter contemplates different positions of the fill ports 150. Which of the fill port 150 orientations is most advantageous will depend on contours of a patient's body. Some embodiments of the fill port block 150 are bendable to allow further matching of the contours of the patient's body.

One of ordinary skill in the art would understand, upon reading and comprehending this disclosure, that various embodiments of the apparatus include various combinations of the illustrated fill port blocks 140, fill port 150 orientations, seal plugs 170, 175, conduit segments 115, 120, 160, sleeve 130 and balloons 110.

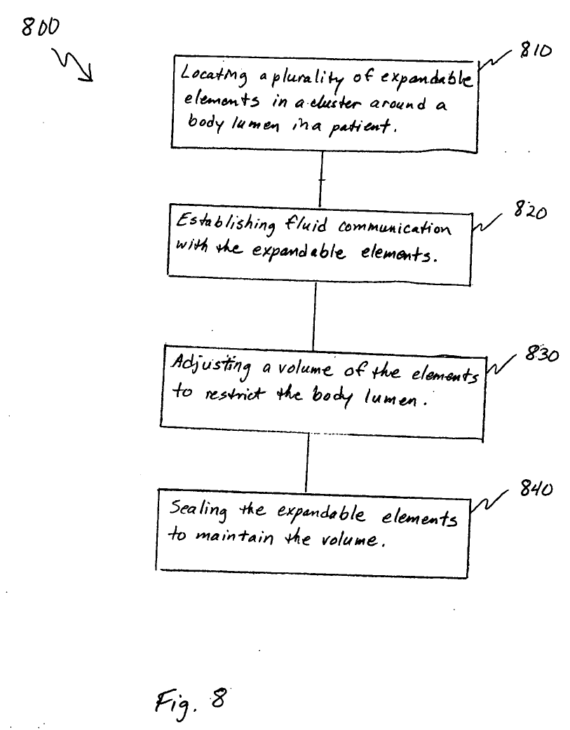

FIG. 8 is a flowchart of a method of providing controllable restriction to a body lumen. In the case where the body lumen is an anal canal, the method 800 provides implantation of a device for fecal continence. At 810, a plurality of expandable elements are located in a cluster around a body lumen, such as the anal canal, of a patient. At 820, fluid communication is established with the expandable elements. In one embodiment, establishing fluid communication includes inserting a hollow needle through a seal plug into a fill port and passing fluid through the hollow needle to adjustably contract or expand a volume of the expandable elements by introducing or removing fluid from the expandable elements. In another embodiment, each element is individually adjustable through a dedicated fill port for each element. At 830, the fluid communication adjusts the volume of the elements to restrict the body lumen. At 840, sealing the expandable elements maintains the volume. In one embodiment the sealing is achieved by using self sealing plugs that re-seal when the hollow needle is withdrawn from the plug.

Although specific examples have been illustrated and described herein, it will be appreciated by those of ordinary skill in the art that any arrangement calculated to achieve the same purpose could be substituted for the specific example shown. This application is intended to cover adaptations or variations of the present subject matter. Therefore, it is intended that the invention be defined by the attached claims and their legal equivalents.

What is claimed is:

1. An apparatus comprising:
a plurality of expandable elements, each of the elements comprised of a membrane of biocompatible material enclosing a volume, wherein the elements expand upon introduction of fluid into the elements and contract upon withdrawal of fluid from the elements, and wherein the expandable elements are adjustable in size by adjusting an amount of fluid in the elements;
a plurality of flexible conduits of biocompatible material providing fluid communication to the expandable elements, wherein a proximal end of each conduit is coupled to each expandable element; and
a fill port block of biocompatible material comprising a plurality of fill ports for providing fluid communication to the expandable elements, wherein a distal end of each conduit terminates on a fill port.

2. The apparatus of claim 1, wherein the expandable elements are adapted and shaped to provide an adjustable volume adjacent a body lumen, and wherein providing an adjustable volume controls restricting of the body lumen.

3. The apparatus of claim 1, wherein each of the plurality of fill ports includes a self sealing plug, wherein the self sealing plug reseals after withdrawal of a needle inserted through the plug for adjusting an amount of fluid in the expandable elements.

4. The apparatus of claim 1, wherein the expandable elements include a lumen shaped to receive a push-wire for tunneling the elements into body tissue of a patient.

5. The apparatus of claim 1, wherein the membrane is comprised of a resiliently elastomeric polymer or polymer blend selected from the group comprising one or both of polyurethane or silicone.

6. The apparatus of claim 1, wherein the expandable elements have an outer surface generally defining an elongate body having semi-spherical end portions.

7. The apparatus of claim 1, wherein the plurality of expandable elements is a first element, a second element and a third element.

8. The apparatus of claim 1, wherein the flexible conduit is tubing comprised of a biocompatible material selected from the group comprising one or both of polyurethane or silicone.

9. The apparatus of claim 1, wherein the apparatus further comprises a strain-relief sleeve for the flexible conduit.

10. The apparatus of claim 1, wherein the fill port block is comprised of a biocompatible metal selected from the group comprising one or both of titanium or tantalum coated with a biocompatible material selected from the group comprising polyurethane or silicone.

11. The apparatus of claim 1, wherein the fill port block is bendable to allow adjusting the block to match contours of the patient's body.

12. The apparatus of claim 3, wherein the fill port block is a tube, wherein the ports are arranged in series along the depth of the tube, and wherein the self-sealing plugs isolate the ports along the tube.

13. The apparatus of claim 1, wherein the fill port block is elongate, and wherein the ports are arranged in parallel such that the flexible tubes are coupled perpendicular to the fill port block.

14. The apparatus of claim 1, wherein the fill port block is elongate, and wherein the ports are arranged in parallel such that the flexible tubes are coupled substantially parallel to a length of the fill port block.

15. A method comprising:
locating a plurality of expandable elements in a cluster around a body lumen of a patient;
establishing fluid communication with the expandable elements; and
adjusting a volume of the elements to restrict the body lumen.

16. The method of claim 15, wherein the locating the plurality of expandable elements results in a generally tulip-shape cluster.

17. The method of claim 15, wherein the locating a plurality of expandable elements includes tunneling at least one element into body tissue of a patient with a push-wire placed in a second lumen of the at least one expandable element.

18. The method of claim 15, wherein establishing fluid communication includes:
inserting a hollow needle through a seal plug into a fill port; and
passing fluid through the hollow needle to adjustably contract or expand a volume of the expandable elements by introducing or removing fluid from the expandable elements.

19. The method of claim 18, wherein the adjusting the volume of the expandable elements includes adjusting post-operatively.

20. The method of claim 15, wherein the body lumen is an anal canal, and wherein restricting the anal canal provides fecal continence.
